(19) United States
(12) Patent Application Publication
(10) Pub. No.: US 2024/0225524 A1
(43) Pub. Date: Jul. 11, 2024

(54) ALLERGY SKIN TEST APPARATUS, METHOD, AND KIT

(71) Applicant: Ahmed Ahmed, Ottawa (CA)

(72) Inventor: Ahmed Ahmed, Ottawa (CA)

(21) Appl. No.: 18/554,332

(22) PCT Filed: Mar. 31, 2022
(86) PCT No.: PCT/CA2022/050495

§ 371 Date: Oct. 6, 2023

Related U.S. Application Data
(60) Provisional application No. 63/185,479, filed on May 7, 2021.

(51) Int. Cl. A61B 5/00

(52) U.S. Cl. CPC A61B 5/411; A61B 2560/0412

(57) ABSTRACT

An apparatus for skin prick testing of immediate allergic reactions by a plurality of applied allergens. The apparatus is provided in three configurations including, respectively, a single head element, an arrangement of two rows of head elements, and an arrangement of three rows of head elements. Use of the apparatus forms the basis for a method of allergy testing, while the apparatus may be provided as a kit for allergy testing, the kit forming an assembly once combined. The kit includes the apparatus in a two or three row configuration, a lower tray with allergen cavities suitable for accepting the head elements of each apparatus, a top tray insert for stable and piloted positioning of each apparatus above the lower tray, and a removeable cover affixable to the lower tray thereby securing the top tray with one or more of the apparatus in an enclosed assembly formed by the kit.

### TECHNICAL FIELD

The present invention relates to allergy testing equipment. More specifically, the present invention relates to an apparatus, method, and kit for skin prick testing of immediate allergic reactions by a plurality of applied allergens.

### BACKGROUND

Allergy testing involves a range of known methods for medical diagnosis of allergies that attempt to provoke a controlled allergic response. Typically, such a controlled response is induced by way of a skin prick test, also called a puncture or scratch test, that determines immediate allergic reactions to as many as fifty different potential allergens at once. This test is usually done to identify allergies to pollen, mold, pet dander, dust mites and foods. In most basic terms, such allergy testing involves a simple, physical pricking of a patient's skin, then inserting a small amount of a substance (i.e., typically an allergen formed a concentrated extract) and awaiting a visual response. If the patient is allergic to the substance, a response including increased redness and swelling will appear and may be accompanied by itchiness or discomfort. The extent of the response may indicate the level of allergic reaction.

Early attempts at skin prick testing involved a rudimentary use of a sharp element such as a needle that was dipped into a potential allergen. The needle was then used to scratch skin and thereby damaging a patient's skin surface sufficiently to introduce the allergen and provoke a reaction. Modern devices operate under the same principle of damaging skin surface while introducing an allergen though such mechanisms vary in the specific devices used. The reaction, if any, will indicate a positive result when a wheal develops. A wheal is a raised white bump surrounded by a small circle of itchy red skin. In general, a large wheal is more likely to indicate a true allergy, although size is not always an accurate predictor. However, if no wheal appears, it is unlikely that an allergic reaction is present.

One prior art device is shown and described in U.S. Pat. No. 5,820,562 granted to Hsiao on Oct. 13, 1998. The Hsiao patent discloses a skin allergy test device that includes a skin allergy test bar and an antigen container. The skin allergy test bar includes a finger grip, an integral cover having a sealing plug portion, an elongated stem extending therefrom, and a plurality of step-shaped punctures, wherein each puncture has a flat step to act as a stop and an integral sharp tip projecting out from the flat step. Each sharp tip has a length shorter than the thickness of the epidermis layer of the skin of human beings such that each puncture will not penetrate the epidermis layer of the skin of human beings due to the flat step of the punctures acting as a stop during a skin allergy test. The container has an inner compartment for storing antigen solution, a top opening for receiving the sealing plug of the skin allergy test bar securely, and a middle opening intercommunicating the inner compartment and top opening. The middle opening has a diameter slightly greater than which of the elongated stem of the skin allergy test bar and is formed with an inner coarse surface for wiping off the antigen solution left on the surface of elongated stem. The Hsiao patent also discloses a raised portion surrounded by the set of shorter punctures. The raised portion is a flat tip that serves both to carry antigen solution and as a stopping mechanism to prevent excessive piercing by the punctures.

Another prior art device is shown and described in U.S. Pat. No. 8,469,900 granted to Hein, Jr. et al. on Jun. 25, 2013. The Hein, Jr. et al. patent discloses an allergy testing system that comprises a skin test device having a grip portion for holding the device. One or more legs extend from the grip, and each leg is oriented to interact with a well containing a potential allergen. Each leg has a test head, and each test head has a plurality of elongated spike members. The elongated spike members have a sharp end configured to receive the potential allergen from a well and to puncture a patient's skin. In addition, each test head has at least one touch activator. The touch activator is longer than the plurality of elongated spike members, such that during an allergy test, the touch activator comes into contact with the skin prior to the elongated spike members, causing the touch activators to activate nerve tissue that blocks transmission of pain, resulting in a reduction of pain and/or discomfort during testing.

Still another prior art device is shown and described in U.S. Pat. No. 8,597,199 granted to Harish et al. on Jun. 25, 2013. The Harish et al. patent discloses an allergy skin test device that purports to cause less pain than commonly used devices that include multi-point sharp puncture heads. The allergy skin test device incorporates multiple dull pressure heads distributed amongst the sharp multi-point puncture heads, each dull pressure head activating a neurological pain gate that reduces pain sensation typically caused by the neighboring multi-point head when it engages the skin. The Harish et al. patent also discloses that the dull pressure heads are slightly longer than the sharp ones, and consequently, the dull pressure heads touch the skin before the sharp heads as the sharp heads are pressed into the skin.

An overriding concept in many prior art devices related to skin allergy testing relates to tactile induced analgesia. This is the phenomenon where concurrent touch and pain on an individual's skin surface reduces the overall intensity of any pain felt by that individual. The basis of this concept was first introduced by Melzack et al. in “Pain Mechanisms: A New Theory” published November 1965 in Science, Volume 150, Issue 3699, pp. 971-979. Melzack et al. established the Gate Control Theory of Pain that predicted that interactions between touch and pain are mostly inhibitory. Subsequent research has shown that pain perception is influenced by touch, with both psychophysical changes and differences in brain activation. Indeed, the intensity of pain reported is consistently reduced in response to touch. This often occurs whether the touch is at the same time as the pain, or even if the touch occurs before the pain. Touch also reduces the activation of cortical areas that respond to painful stimuli.

In light of tactile induced analgesia, prior art skin allergy testing devices have been configured to include a tactile element which engages skin prior to engagement by a skin piercing element. However, in such configurations, prior art devices have failed to recognize the skin stretching effect of tactile elements and the impact such deformation of skin has on pain incurred by skin piercing elements.

### SUMMARY

The present invention provides an apparatus, method, and kit for skin prick testing of immediate allergic reactions by a plurality of applied allergens. The invention utilizes tactile induced analgesia in a manner that reduces the negative effect of skin stretching created by tactile elements. The present invention accomplishes reduced pain by providing a configuration including a plurality of tactile elements which engage a patient's skin surface uniformly and just after engagement by a plurality of skin piercing elements. In accordance with the present invention, the skin stretching effect of tactile elements is substantially eliminated prior to initial skin piercing.

It should be understood that mention of skin piercing in the context of the present invention is intended to connote only penetration of the outermost epithelial layer of a patient sufficient to enable introduction to the patient's skin of a liquified allergen held upon the piercing elements by capillary action.

The present invention provides an apparatus for skin prick testing of immediate allergic reactions by a plurality of applied allergens. The apparatus is provided in three configurations including, respectively, a single head element, an arrangement of two rows of head elements, and an arrangement of three rows of head elements. Use of the apparatus forms the basis for a method of allergy testing, while the apparatus may be provided in terms of a kit for allergy testing. The kit includes the apparatus in either a two or three row configuration, a lower tray with allergen cavities suitable for accepting the head elements of each apparatus, a top tray insert for stable and piloted positioning of each apparatus above the lower tray, and a removable cover affixable to the lower tray thereby securing the top tray with one or more of the apparatus in an enclosed assembly formed by the kit.

In a first aspect, the present invention provides an assembly for allergy testing, the assembly comprising: at least one apparatus having a plurality of head elements, each head element including four pressure tips interspersed alternatingly between four piercing tips and together arranged in a circular pattern within which circular pattern a fifth piercing tip is centrally located: a lower tray including a plurality of well sections for retaining a plurality of allergens, the well sections including a plurality of wells corresponding to the plurality of head elements: a cover for removable attachment to the lower tray: and a top tray for retaining the apparatus: wherein the cover, upon attachment to the lower tray, retains the top tray and the at least one apparatus therebetween.

In a second aspect, the present invention provides an apparatus for allergy testing, the apparatus comprising: a head element including four pressure tips interspersed alternatingly between four piercing tips and together arranged in a circular pattern within which circular pattern a fifth piercing tip is centrally located.

In a third aspect, the present invention provides a kit for allergy testing, the kit comprising: at least one apparatus having a plurality of head elements, each head element capable of providing four pressure tips interspersed alternatingly between four piercing tips and together arranged in a circular pattern within which circular pattern a fifth piercing tip is centrally located: a lower tray capable of providing a plurality of well sections for retaining a plurality of allergens, the well sections providing a plurality of wells corresponding to the plurality of head elements: a cover capable of secure attachment to the lower tray: and a top tray capable of securely situating the apparatus: wherein the cover is capable of removable attachment to the lower tray and situating the top tray along with the apparatus therebetween.

In a fourth aspect, the present invention provides a method of administering an allergy test by means of an apparatus having at least one head element including a plurality of piercing tips and a plurality of rounded pressure tips slightly shorter than the plurality of piercing tips, the method comprising: placing the head element into at least one well containing an allergen solution to load the allergen solution onto the head element: and administering the allergen solution to a skin surface of a patient by pressing the apparatus against the skin surface such that the rounded pressure tips of the head element contacts with the skin immediately after the piercing tips of the head element punctures the skin of the patient to thereby deliver the allergen solution: wherein the pressure tips activate nerve tissue of the patient to suppress pain thereby resulting in a substantial elimination of pain during testing.

In a fifth aspect, the present invention provides an apparatus, method, and kit as shown and described within the accompanying specification and drawings.

### BRIEF DESCRIPTION OF THE DRAWINGS

The present invention will now be described by reference to the following figures, in which identical reference numerals refer to identical elements.

FIG. 1 is an expanded perspective view of a complete inventive assembly showing a duo-configuration of an apparatus in accordance with one embodiment of the present invention.

FIG. 2 is a perspective view of the assembly of FIG. 1 shown in a closed position.

FIG. 3 is a top view of the assembly of FIG. 2.

FIG. 4 is a bottom view of the assembly of FIG. 2.

FIG. 5 is a front-side edge view of the assembly of FIG. 2.

FIG. 6 is a right-end edge view of the assembly of FIG. 2.

FIG. 7 is a cross-sectional view of the assembly taken along line 7-7 shown in FIG. 5.

FIG. 8 is a perspective view of the lower tray of the assembly shown in FIG. 1.

FIG. 9 is a top view of the lower tray of FIG. 8.

FIG. 10 is a bottom view of the lower tray of FIG. 8.

FIG. 11 is a front-side edge view of the lower tray of FIG. 8.

FIG. 12 is a right-end edge view of the lower tray of FIG. 8.

FIG. 13 is a cross-sectional view of the lower tray taken along line 13-13 shown in FIG. 9.

FIG. 14 is a partial cross-sectional view of the of the lower tray indicated by detailed portion 14 shown in FIG. 13.

FIG. 15 is a perspective view of the cover of the assembly shown in FIG. 1.

FIG. 16 is a top view of the cover of FIG. 15.

FIG. 17 is a bottom view of the cover of FIG. 15.

FIG. 18 is a front-side edge view of the cover of FIG. 15.

FIG. 19 is a right-end edge view of the cover of FIG. 15.

FIG. 20 is a cross-sectional view of the cover taken along line 20-20 shown in FIG. 16.

FIG. 21 is a perspective view of the top tray of the assembly shown in FIG. 1.

FIG. 22 is a top view of the top tray of FIG. 21.

FIG. 23 is a bottom view of the top tray of FIG. 21.

FIG. 24 is a front-side edge view of the top tray of FIG. 21.

FIG. 25 is a right-end edge view of the top tray of FIG. 21.

FIG. 26 is a cross-sectional view of the top tray taken along line 20-20 shown in FIG. 22.

FIG. 27 is a perspective view of a duo-configuration apparatus of the assembly shown in FIG. 1.

FIG. 28 is a top view of the apparatus of FIG. 27.

FIG. 29 is a bottom view of the apparatus of FIG. 27.

FIG. 30 is a front-side edge view of the apparatus of FIG. 27.

FIG. 31 is a right-end edge view of the apparatus of FIG. 27.

FIG. 32 is a bottom view of the head element of the apparatus indicated by detailed portion 33 shown in FIG. 31.

FIG. 33 is an enlarged view of the head element of the apparatus indicated by detailed portion 33 shown in FIG. 31.

FIG. 34 is a perspective view showing a trio-configuration of another apparatus in accordance with a second embodiment of the present invention.

FIG. 35 is a bottom view of the apparatus of FIG. 35.

FIG. 36 is a perspective view showing a mono-configuration of another apparatus in accordance with a third embodiment of the present invention.

FIG. 37 is a top view of the apparatus of FIG. 36.

FIG. 38 is a bottom view of the apparatus of FIG. 36.

FIG. 39 is a front view of the apparatus of FIG. 36.

FIG. 40 is a side view of the apparatus of FIG. 36.

FIG. 41 is an enlarged view of the head element of the apparatus indicated by detailed portion 42 shown in FIG. 40.

FIG. 42 is an enlarged view of the head element as shown in FIG. 38.

FIG. 43 is a top view of an alternative embodiment of the apparatus in FIG. 28.

### DETAILED DESCRIPTION

The present invention provides an apparatus for skin prick testing of immediate allergic reactions by a plurality of applied allergens. The apparatus is provided in three configurations: mono, duo, and trio, each of which corresponds, respectively, to a single head element, an arrangement of two rows of head elements, and an arrangement of three rows of head elements. Relatedly, use of the inventive apparatus forms the basis for an innovative method of allergy testing. Likewise, the inventive apparatus may be provided in terms of a kit for allergy testing. The kit preferably includes the inventive apparatus in either a duo or trio configuration, a lower tray with allergen cavities suitable for accepting the head elements of each apparatus, a top tray insert for stable and piloted positioning of each apparatus above the lower tray, and a removable cover affixable to the lower tray thereby securing the top tray with one or more of the apparatus in an enclosed assembly formed by the kit. The inventive apparatus, method, and kit will now be described in more detail with regard to the figures.

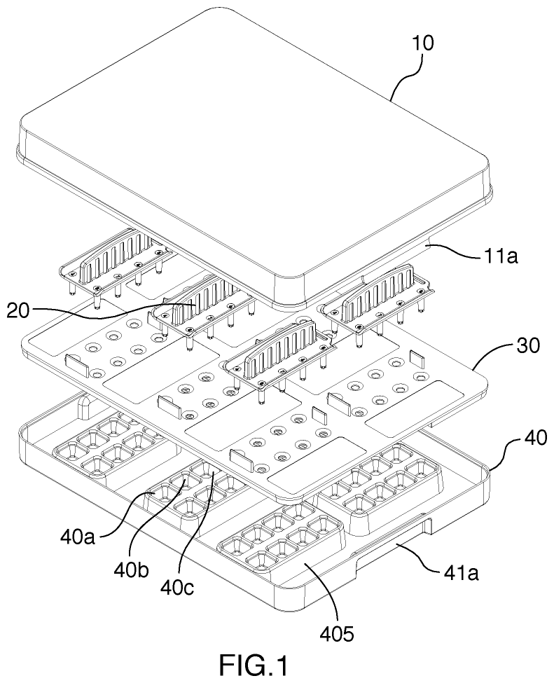

Referring now to FIG. 1, one embodiment of the present invention is shown in an expanded perspective view. This illustrates a complete inventive assembly that may be provided in a kit form including constituent parts intended for use together. In particular, the assembly shown includes four types of parts: cover 10, apparatus 20, top tray 30, and lower tray 40. The cover 10 is provided and is attachable to the lower tray 40 in a clamshell manner. The top tray 30 is provided and securely fits within the lower tray 40. As well, one or more apparatus 20 in accordance with the present invention are provided. Here, a duo-configuration apparatus 20 is shown, four of which are visible and two are hidden from view.

Each of the constituent parts of the assembly shown in FIG. 1 are preferably fabricated from a suitably durable material such as, but not limited to, injection molded plastic. As mentioned, the cover 10 is provided and is attachable to the lower tray 40 in a clamshell manner. This is made possible by a lower peripheral edge of the cover 10 that overlaps with a top peripheral edge of the lower tray 40. The cover 10 is snap-fit to the lower tray 40 by way of tabs 11a interlocking with recesses 40a provided in pairs (only one visible), each pair located on the right and left sides of the assembly.

The lower tray 40 includes well sections 405 that are integrated into the lower tray 40. Each well section 405 includes a plurality of wells 40a, 40b, 40c, etc. where the number of wells in each well section corresponds to the particular apparatus 20. Here, each apparatus as shown includes eight head elements (described further below). Thus, each well section as shown also includes eight wells.

As discussed, the assembly of FIG. 1 is provided in the form of a “dry” kit of constituent parts whereby a user (e.g., an allergist or other appropriate medical professional) of the inventive assembly may assemble the constituent parts and add different allergens to the wells.

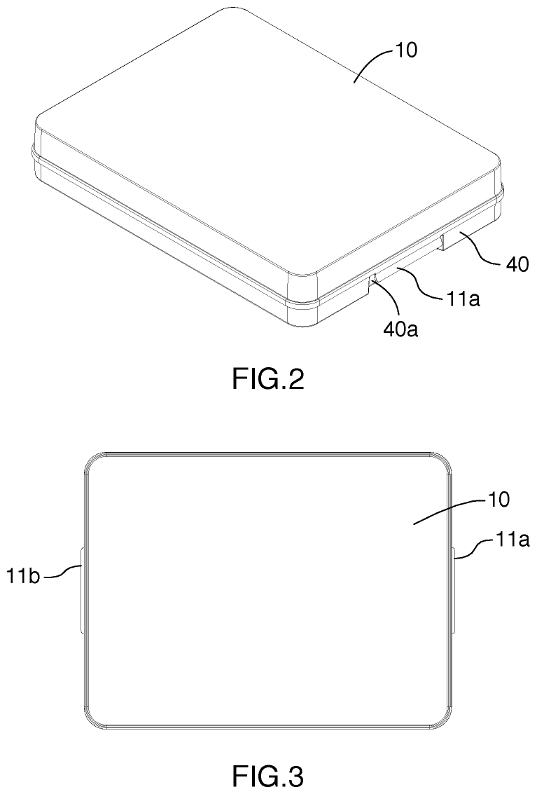

Referring now to FIG. 2, the assembly of FIG. 1 is shown in a closed position. Here, the snap-fit attachment is viewable from the right end of the assembly, though it should be readily understood that both ends include the snap-fit attachment whereby tab 11a interlocks with recess 41a to maintain the closed position of the cover 10 atop the lower tray 40. From FIG. 3, the top view of the assembly in the closed position shows the right-side tab 11a along with a corresponding left-side tab 11b which are essentially mirror images of one another in both form and function.

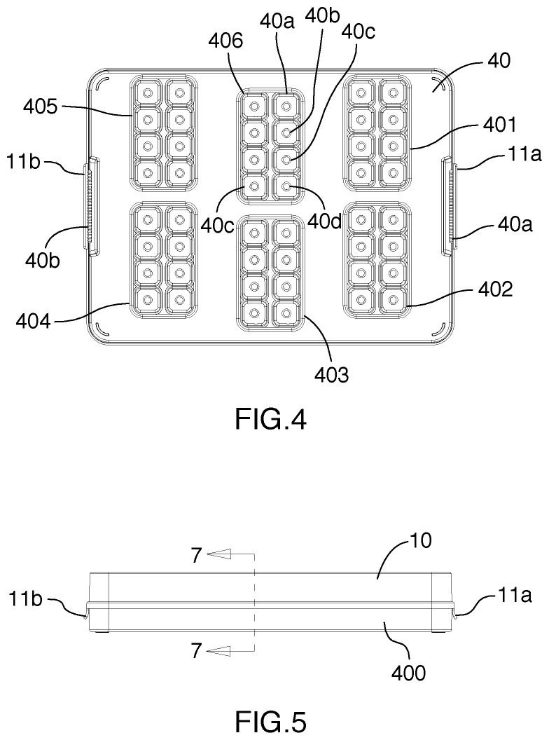

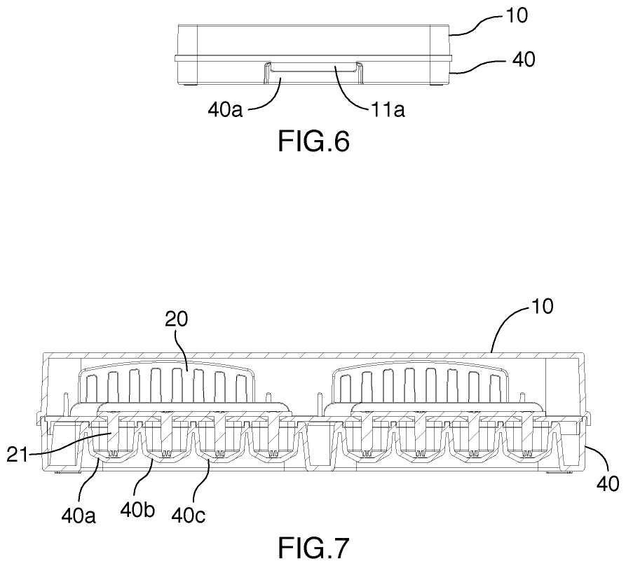

Referring now to FIG. 4, a bottom view of the assembly in the closed position is shown. As similarly described above, the assembly in the closed position shows the right-side tab 11a along with a corresponding left-side tab 11b which each respectively interlock with recesses 40a and 40b. Well sections 401, 402, 403, 404, 405, and 406 are visible molded into the body of the lower tray 40. FIGS. 5 and 6 each further illustrate, respectively, front-side and right-side edge views of the assembly and shows the interlocking nature of the cover with the lower tray via the tabs and recesses.

With further reference to FIG. 4, each well section (e.g., 406) includes a set of wells (e.g., 40a, 40b, 40c, 40d, 40e, etc.). For illustrative clarity, all wells are not labeled. It should be understood that all wells are intended to be identical and suitably dimensioned to allow an amount of allergen to be held therewithin that is of a suitable volume for allergy testing purposes. Raised feet may be provided at the exterior bottom corners of the lower tray to assist in placement of the assembly on a flat surface such as a table. The feet may be formed in any manner such as being integrated during the molding process of the lower tray or later added after formation of the lower tray. Likewise, the feet may be preferably formed of a suitable material such as a high tack rubber to prevent movement of the assembly once placed atop a flat surface such as a table.

Referring now to FIG. 7, there is illustrated a cross-sectional view of the assembly taken along line 7-7 shown in FIG. 5. Here, head elements 21 of each apparatus 20 are clearly visible with their tips extending into each well (e.g., 40a, 40b, 40c, etc.). The tips will be further described in more detail hereinbelow. FIG. 7 serves to illustrate the compact nature of the assembly such that the assembly efficiently retains each apparatus within a corresponding well section between the clamshell-like enclosure formed by the cover 10 and lower tray 40.

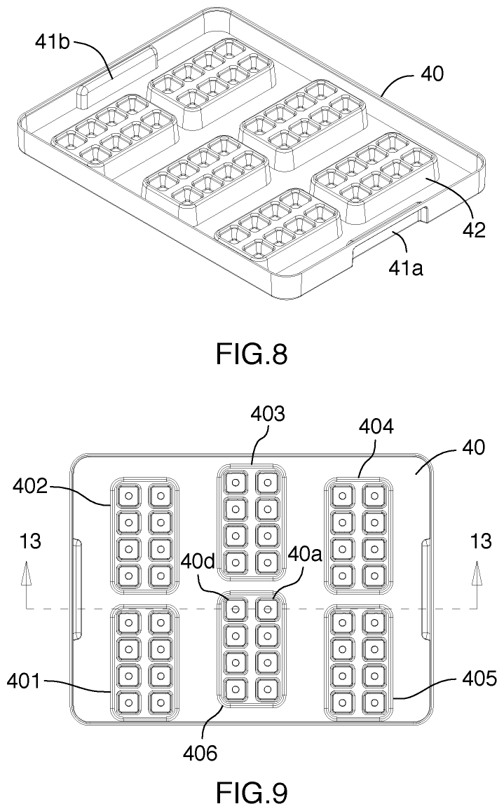

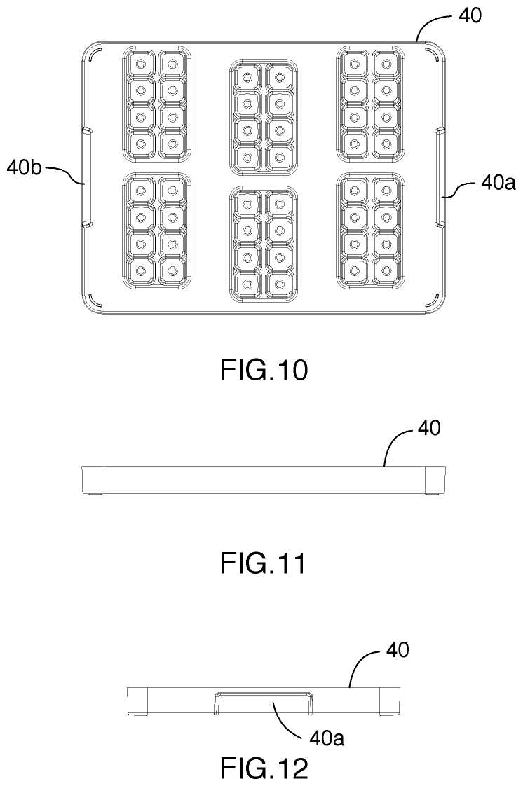

Referring now to FIG. 8, a perspective view of the lower tray 40 of the assembly shown in FIG. 1 is illustrated. Here, the well sections (e.g., 404) are clearly shown as are the recesses 41a and 41b. As previously mentioned, the lower tray 40 may be molded by a single material such as, but not limited to, plastic or any suitably durable material. FIG. 9 is a top view of the lower tray 40 of FIG. 8. Here, the lateral displacement of well sections 403 and 406 is visible relative to well sections 401, 402, 404, and 405. Such lateral displacement is useful in terms of enabling a user to visually recall accurately the identification of the particular well section due to the fact that lateral displacement pattern differs when the lower tray 40 is rotated by 180 degrees. Thus, if the assembly is moved from one location to another during use, there is a clear orientation of the assembly that may be easily recognizable by the user. This advantageously helps prevents a user from inadvertently rotating the assembly and losing track of which well section is which. Such lateral displacement of well sections is also clearly visible by way of FIG. 10 which is a bottom view of the lower tray of FIG. 8. Front-side edge and right-end edge views of the lower tray 40 are shown, respectively, by way of FIG. 11 and FIG. 12.

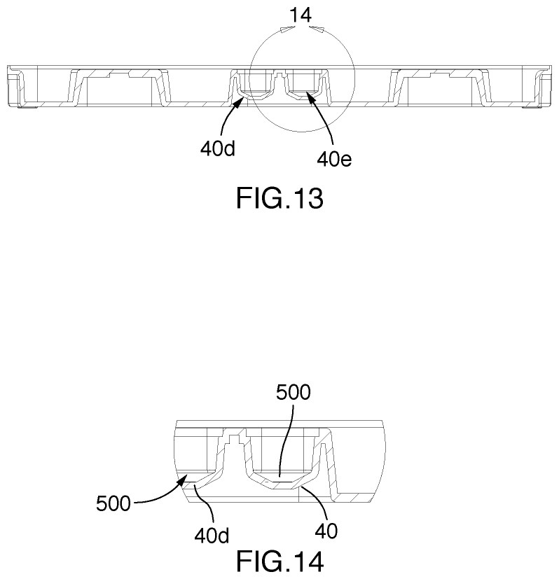

Referring now to FIG. 13, the lower tray is shown in cross-section where the view represents an edgewise cross-sectional view of the lower tray taken along line 13-13 shown in FIG. 9. Here, cross sections of the wells 40d and 40e are visible. Likewise, FIG. 14 is a partial cross-sectional view of the of the lower tray indicated by detailed portion 14 shown in FIG. 13. In this detailed view, each of wells 40d and 40e show an allergen storage space 500 suitably sized to hold at least a medically significant volume of allergen. It should be understood that the determination of what constitutes a medically significant volume of allergen is subject to the given concentration of the allergen and may vary in accordance with the implementation of the present invention. More specifically, the well volumes of the present invention are preferably sized to hold up to 2.5 ml of allergen, thus advantageously reducing the need for an allergist to frequently refill the wells.

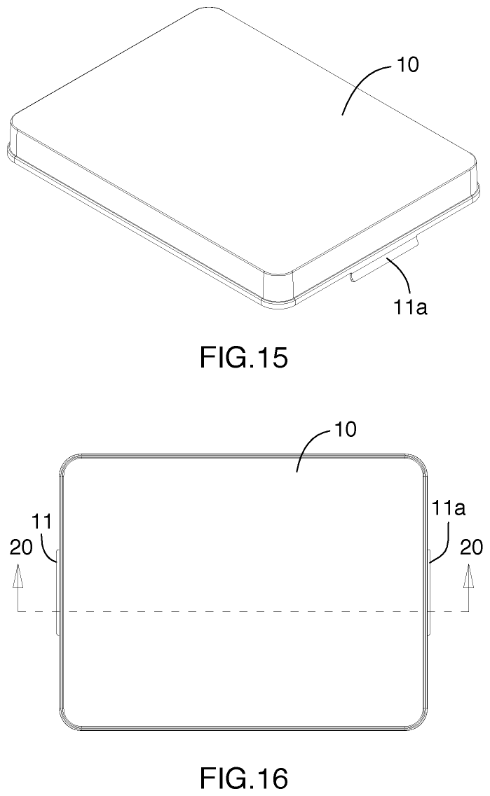

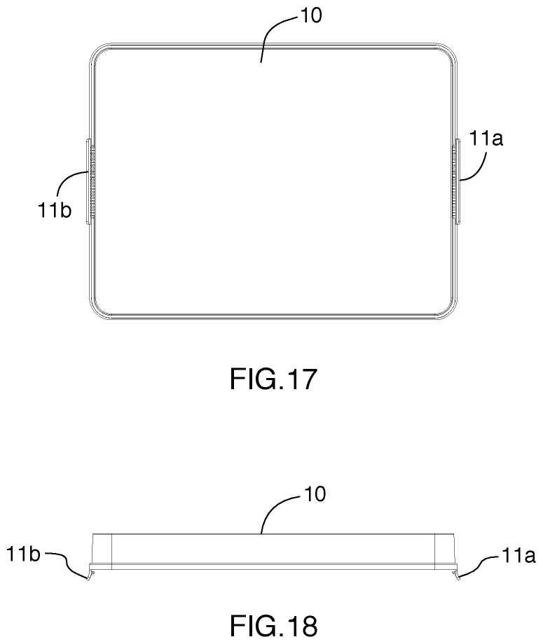

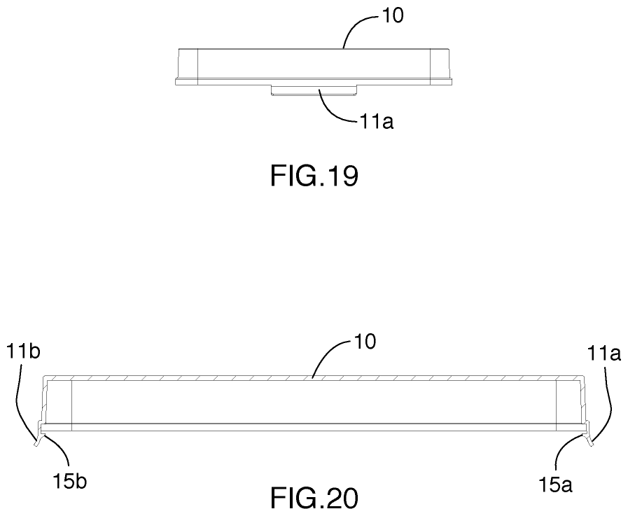

Referring now to FIG. 15, the cover 10 of the assembly shown in FIG. 1 is illustrated and shows the rightmost tab 11a. As previously mentioned, the cover 10 may be fabricated by any suitable method and material such as, but not limited to, injection molded plastic. Likewise, the cover 10 may be molded simultaneously with raised lettering and/or logos. Still further, the cover 10 may be fabricated from an opaque or clear material. It should be understood that a clear material may be rendered opaque in areas not desired to be clear by methods such as abrading or the like. Such partially obscured sections of the cover may therefore form “windows” whereby the apparatus may be viewable therethrough. It should be understood that any combination of opacity may therefore be fabricated without straying from the intended scope of the present invention. FIGS. 16, 17, 18 and 19 are, respectively, top, bottom, front-side edge, and right-side edge views of the cover 10 of FIG. 15.

Referring now to FIG. 20, the cover 10 is illustrated in cross-section taken along line 20-20 shown in FIG. 16. Here, tabs 11a, 11b are visible along with locking mechanisms 15a, 15b. Due to the size, shape, and material, the tabs 11a, 11b are flexible in such a manner that they may be deflected when the cover is snap-fit over the corresponding recesses of the lower tray as previously seen in FIG. 7.

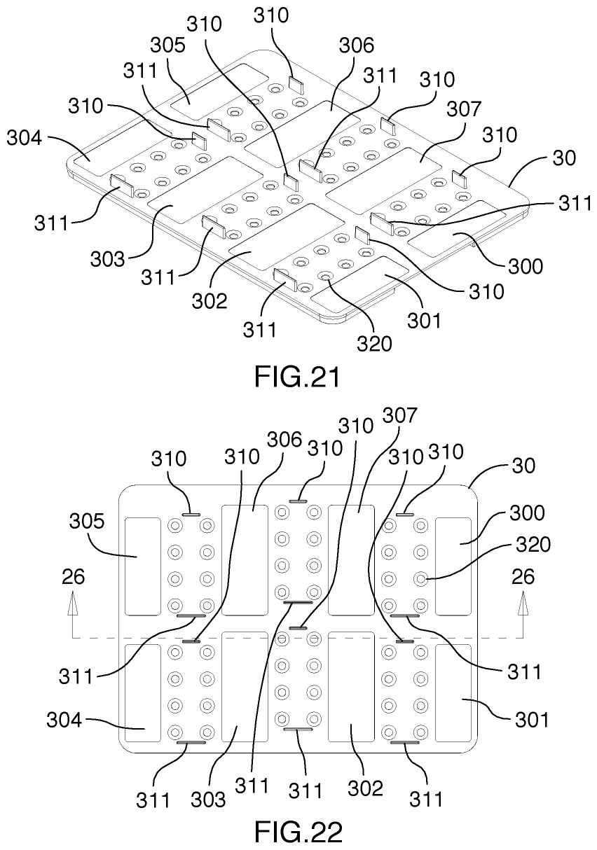

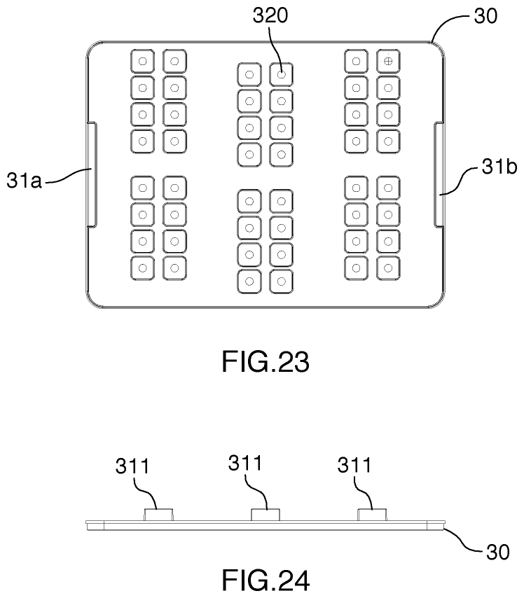

Referring now to FIG. 21, there is illustrated a perspective view of the top tray 30 of the assembly shown in FIG. 1. The top tray 30 provides several useful functions as an intermediate structure between the lower tray 40 located below the top tray 30 and the plurality of apparatus 20 that are generally located above the top tray 30. FIG. 22 is a top view of the top tray of FIG. 21, while FIG. 23 is a bottom view of the top tray of FIG. 21.

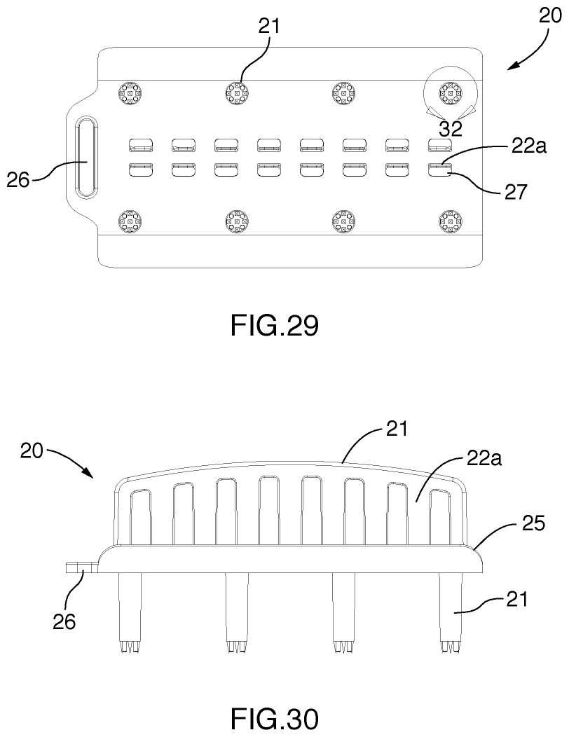

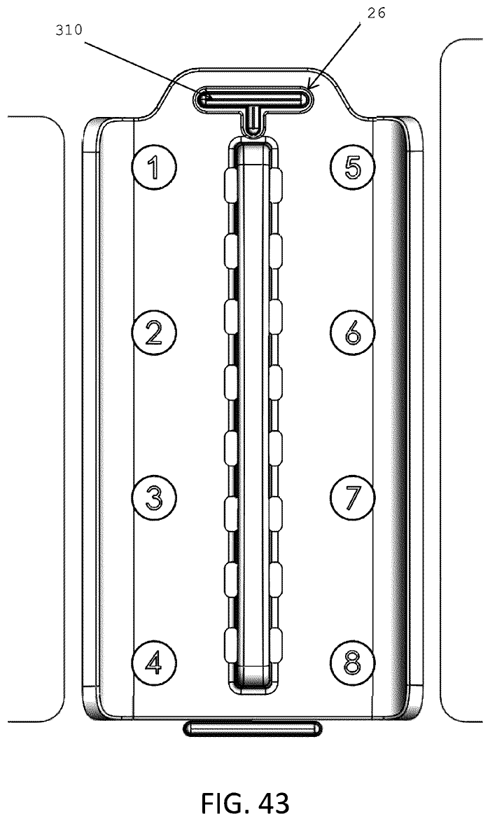

A first function of the top tray 30 is to provide a secure seat for each apparatus 20. This is accomplished by a combination of structures integrated into the top tray 30. These structures include apertures 320 arranged in a pattern to match the number of head elements. Each aperture 320 includes an inner dimension sufficient to allow passage therethrough of each head element. Each aperture 320 includes a peripheral edge that is beveled in such a manner so as to guide, or pilot, the head elements into the corresponding opening that forms each aperture 320. Each apparatus (via retention hole 26 as shown and described further below with regard to FIG. 29) also is further guided, or piloted, and subsequently held in place atop the top tray 30 by way of a retention bar 310 suitably sized for placement within retention hole 26. Likewise, stops 311 are provided opposite each retention bar 310. Each stop 311 and corresponding retention bar 310 form space between which each apparatus 20 is thus seated. Because stops 311 are less wide than the retention bars, confusion as to the orientation of the apparatus is substantially eliminated when placed therebetween by a user. It should be clear that retention bar 310, while illustrated as a straight bar in the Figures, may have a configuration other than as a straight bar. In one implementation, the retention bar 310 has a T-shaped configuration and the retention hole 26 is also suitably configured to have a T-shaped configuration such that the retention bar 310 suitably mates with the retention hole 26. The T-shaped configuration of the retention bar 310 and of the retention hole 26 can be seen in FIG. 43.

Another function of the top tray 30 is to provide a covering above the well sections and to thereby maintain the integrity of the allergens placed into each well.

Still another function of the top tray 30 is to provide a series of indicator areas 300, 301, 302, 303, 304, 305, 306, and 307. Each indicator area provides a location for labeling that indicates the particular allergen in an adjacent well. The indicator areas may alternatively be surfaced in such a manner so as to allow a user to write on the surface of the indicator area. Still further, each indicator area may be color coded for a variety of different types of allergens. Overall, it should be understood that the indicator areas are easily and advantageously customizable by a user in any manner suitable for the given implementation of the present invention without straying from the inventive concept.

With further reference to FIG. 23 showing the bottom view of the top tray, there are shown thinned edge areas that form gaps 31a and 31b. Each gap is suitably shaped to enable the top tray 30 to firmly rest upon the interior protrusions formed by recesses 41a, 41b (best seen in FIG. 8). Once in place atop the lower tray 40, lateral movement of the top tray 30 in any direction is substantially precluded.

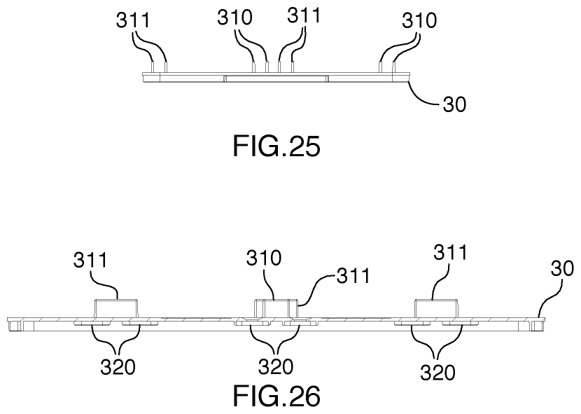

With reference to FIG. 24, the front-side edge view of the top tray 30 is shown. Here, the stops 311 are visible such that they extend from the surface of the top tray 30. Likewise, FIG. 25 is a right-end edge view of the top tray 30 where both the stops 311 and retention bars 310 are visible extending from the surface of the top tray. As previously mentioned, the top tray 30 may be fabricated by any suitable manner and material such as, but not limited to, injection molded plastic. In such instance, the stops 311 and retention bars 310 are preferably integrated with the surface structure that forms the rest of the top tray 30. Thicknesses of the stops 311 and retention bars 310 are preferably chosen to reduce instances of breakage of the stops 311 and retention bars 310.

With reference to FIG. 26, a cross-sectional view is provided and illustrates the cross section of the top tray 30 as taken along line 20-20 shown in FIG. 22. Here, each aperture 320 is visible in relation to the stops 311 and retention bars 310.

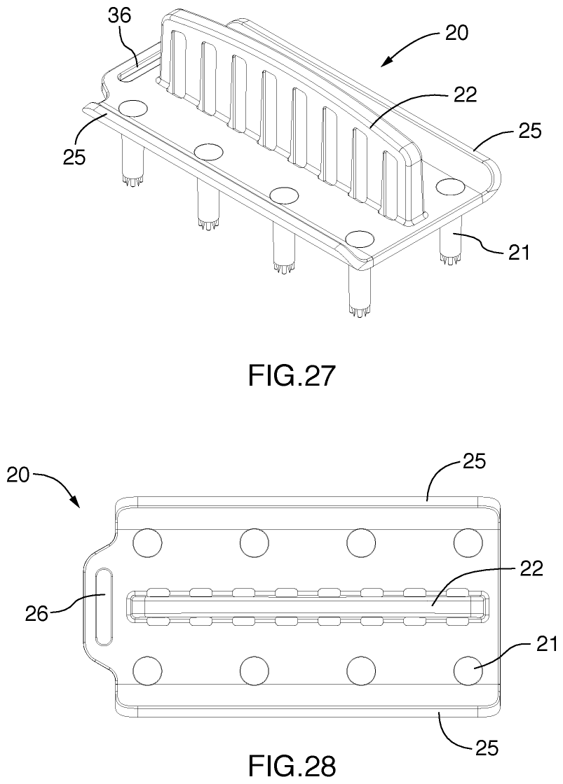

With reference to FIG. 27, there is illustrated a perspective view of a duo-configuration apparatus 20 of the assembly shown in FIG. 1. As previously described, a duo-configuration includes two rows of head elements 21. The head elements may be formed integrally with the rest of the apparatus 20, or more preferably, formed separate from the rest of the apparatus 20. Because of the manufacturing tolerances required at the tips of each head element, fabricating the head elements 21 separately may be useful. In such instance, each head element may be fabricated and then subsequently bonded to a corresponding hole in the surface of the body of the apparatus 20. Such bonding may be accomplished by any known manner such as, but not limited to, vibration welding of constituent plastic parts. An alternative manner of manufacture may involve fabricating both sharp and blunt portions of the head elements concurrently through one or more known methods including, but not limited to, insert molding, overmolding, injection molding, or machining.

With continued reference to FIG. 27, the apparatus 20 is shown to include a flared edge forming wings 25. Formation of the wings 25 increases the overall structural integrity of the apparatus 20 such that deformation (e.g., lengthwise twisting or overall flexing) of the overall structure of the apparatus 25 during use is substantially eliminated. FIGS. 28 and 29 illustrate, respectively, top and bottom views of the apparatus 20. In each illustration, the aforementioned retention hole 26 is clearly visible in an extended area of the surface of the apparatus 20. A handle 22 is also provided in a spine-like manner along the length of the apparatus 20. For ease of manufacture, the handle 22 is preferably fabricated separately from the rest of the apparatus 20. However, the handle may be injection molded together with the rest of the apparatus. The handle 22 includes protrusions 22a which are flexible extensions of the handle formed to engage corresponding openings (visible in FIG. 29 as item 27). The protrusions 22a engage with openings 27 in a manner such that the handle 22 may snap into place atop the surface of the apparatus 20 and thus be retained thereupon. FIG. 30 further shows the apparatus 20 from a front-side edge view: Here, the alignment of the head elements is illustrated such that the two rows are clearly aligned adjacent one another.

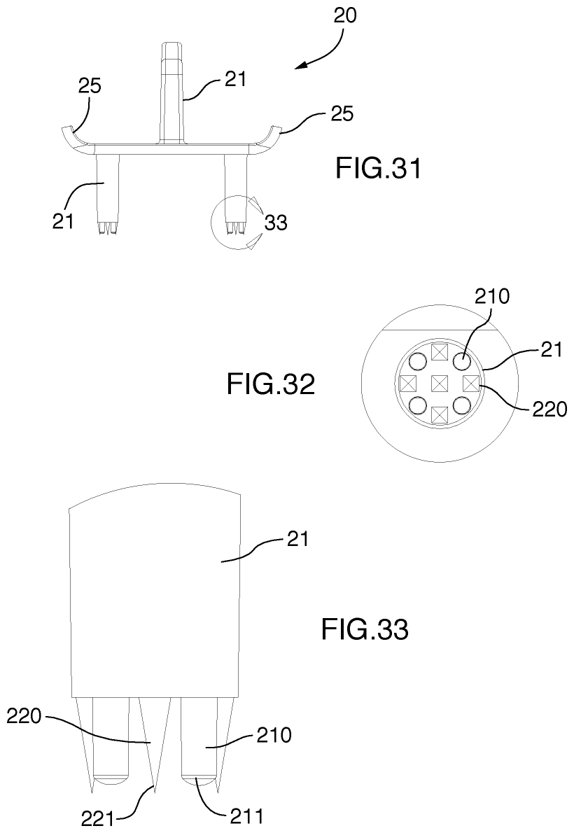

With reference to FIG. 31, there is illustrated a right-end edge view of the apparatus 20 shown in FIG. 27. Again, the wings 25 are visible along with the lengthwise alignment of the head elements 21. FIG. 32 is a bottom view of the head element 21 of the apparatus indicated by detailed portion 33 shown in FIG. 31. Here, there are shown four pressure tips 210 and five piercing tips 220. FIG. 33 is an enlarged view of the head element 21 of the apparatus 20 indicated by detailed portion 33 shown in FIG. 31. Here, the lengthwise relationship between the pressure tips 210 and the piercing tips 220 is clearly visible such that the piercing tips 220 extend slightly longer than the pressure tips. The ends of the piercing tips 220 each includes a sharp point 221.

An advantage of the present invention resides in the relationship between the pressure tips 210 and the piercing tips 220. Because each piercing tip 220 extends slightly beyond the ends 211 of the pressure tips 210, the piercing tips 220 engage a patient's skin surface just prior to the end of the pressure tips 210 engaging the patient's skin surface. This advantageously creates a tactile induced analgesia effect by the pressure tips 210 that occurs substantially concurrent with the piercing of the skin surface by the piercing tips 220. Moreover, this tactile induced analgesia effect occurs without any related stretching of the patient's skin surface. Thus, allergy testing is accomplished in a substantially less painful manner.

The tactile induced analgesia effect is further enhanced by the particular configuration of the plurality of pressure tips 210 and piercing tips 220. Specifically, the pressure tips 210 are interspersed alternatingly between piercing tips 220. This effectively prevents a user from inadvertently piercing a patient's skin surface too deeply before contact with the pressure tip 210 is made if, for example, the apparatus is placed upon the patient's skin at an angle rather than squarely set atop the patient's skin surface. Still further, it should be noted that the end 211 of each pressure tip 210 is rounded. Such rounded end 211 ensures adequate pressure against a patient's skin surface while further precluding undue stretching of the skin surface. Yet still further, the function of the inventive apparatus is enhanced by the presence of five piercing tips 220 including one centrally located at the tip and four located around the periphery of the head element 21. Thus, it has been determined that an optimal configuration includes four pressure tips 210 interspersed alternatingly between four piercing tips 220 and together arranged in a circular pattern within which circular pattern a fifth piercing tip 220 is centrally located. Such optimal configuration increases the desired tactile induced analgesia effect while reducing stretching of skin surface and substantially reduces pain sensed by the patient.

It should be understood that while five piercing tips and four pressure tips have been shown and described, any number of such tips may be provided without straying from the intended scope of the present invention so long as the pressure tips are interspersed alternatingly between the piercing tips around the periphery of the head element. Likewise, the periphery may be substantially circular as shown or any relatively round shape such as, but not limited to, octagonal, hexagonal, . . . etc.

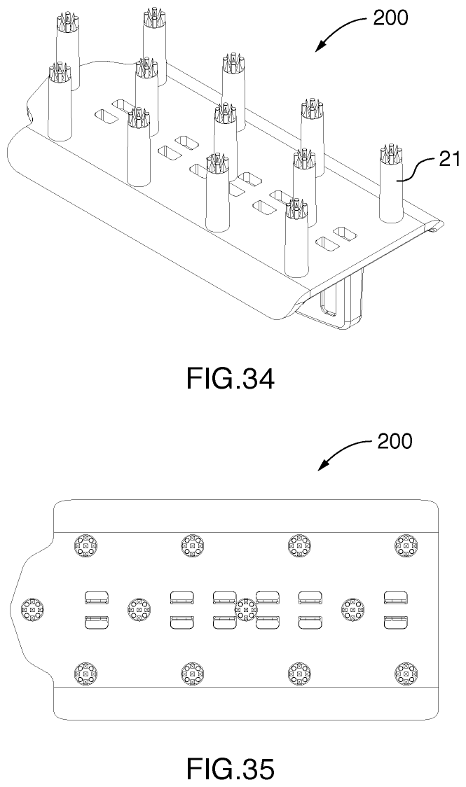

With reference to FIG. 34, a second embodiment of the present invention is shown in the form of an apparatus having a trio-configuration. FIG. 35 is a bottom view of the apparatus of FIG. 35. In this second embodiment, three rows of head elements 21 are provided which results in an apparatus 200 that includes a total of twelve head elements 21. Advantageously, the middle row of head elements 21 is displaced lengthwise. This configuration enables a user to easily determine the orientation of the apparatus 200 due to the unique arrangement of the staggered head elements 21. It is further an advantage that the distance between the head elements on the sides is the same distance diagonally between the head elements located on the sides and the closest diagonal middle row head element.

The trio-configuration in accordance with the present invention would, of course, require corresponding alternations of the well sections of the lower tray along with corresponding alterations of the apertures of the top tray. Such alterations need not be further described herein as modifications should be apparent to one skilled in the injection molding art in light of the detailed description above regarding the first embodiment.

In terms of the second embodiment, one advantage of such trio-configuration shown in FIGS. 34 and 35 is that an apparatus embodying the trio-configuration provides an optimal use of the space on adult arms and on the backs of both adults and children to test more allergens. Still further, the staggering of head elements in the trio-configuration substantially eliminates cross contamination of allergens when wiping the allergen extracts subsequent to use of the apparatus.

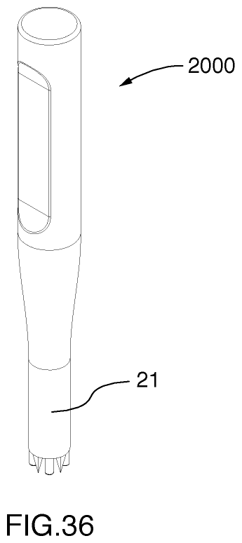

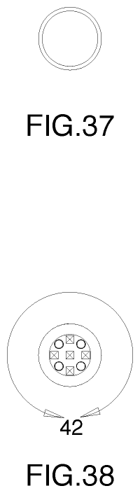

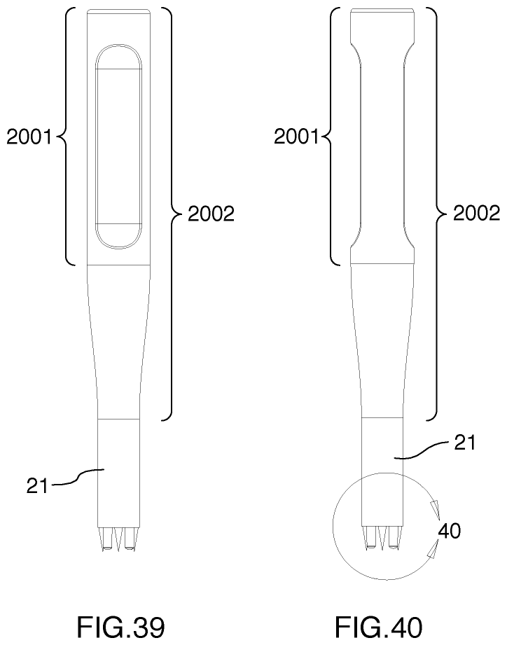

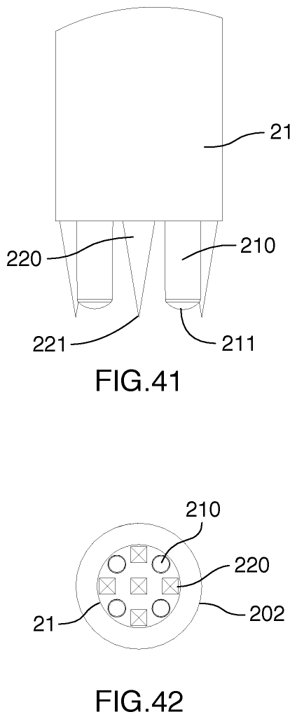

A third embodiment of the present invention is shown and described with reference to FIGS. 36 though 42. In particular, FIG. 36 is a perspective view showing the mono-configuration of the apparatus in accordance with the third embodiment of the present invention. Here, the mono-configuration apparatus 2000 is illustrated. Such mono-configuration may be utilized in conjunction with a simple tray (not shown) of allergen into which the single head element 21 is dipped. FIGS. 37, 38, 39, and 40 are, respectively, top, bottom, front, and side views of the apparatus 2000 of FIG. 36. The mono-configuration apparatus 2000 includes a handle section 2001 of its main body 2002 which is attached to a head section 21. The head section 21 is as described above with regard to the previous embodiments. FIG. 41 is an enlarged view of the head element 21 of the apparatus 2000 indicated by detailed portion 42 shown in FIG. 40. Likewise, FIG. 42 is an enlarged view of the head element 21 as shown in FIG. 38. As before, the lengthwise relationship between the pressure tips 210 and the piercing tips 220 is clearly visible such that the piercing tips 220 extend slightly longer than the pressure tips.

It should be understood that the present invention of the first and second embodiments discussed above may be provided in the form of a kit. The kit may include a cover capable of secure attachment to a lower tray. The lower tray provides a plurality of well sections capable of retaining a plurality of allergens. The allergens are provided separate from the well sections in suitable dispensing containers (e.g., dropper vial) capable of filling each well. The kit further includes a plurality of apparatus capable of providing four pressure tips interspersed alternatingly between four piercing tips and together arranged in a circular pattern within which circular pattern a fifth piercing tip is centrally located. Such optimal configuration of pressure tips and piercing tips capable of increasing a desired tactile induced analgesia effect while reducing stretching of skin surface and thereby substantially reducing pain sensed by the patient during allergy testing while providing enough piercing points to trigger an allergic response among allergic individuals. The kit further includes a top tray capable of securely situating the plurality of apparatus. The kit further includes a cover capable removable attachment to the lower tray and situating the top tray along with the plurality of apparatus therebetween.

In operation, the embodiments of the apparatus in accordance with the present invention are useful in conjunction with a method for administering an allergy test with an apparatus having at least one head element including a plurality of piercing tips and a plurality of rounded pressure tips slightly shorter than the plurality of piercing tips, the method including the steps of placing the head element into at least one well containing an allergen solution to load the allergen solution onto the head element: administering the allergen solution to skin of a patient by pressing the apparatus against the skin such that the rounded pressure tips of the head element comes into contact with the skin immediately after the piercing tips of the head element punctures the skin of the patient and delivers the allergen solution, wherein the pressure tips activate the patient's nerve tissue to suppress pain thereby resulting in a substantial reduction of pain during testing.

A person understanding this invention may now conceive of alternative structures and embodiments or variations of the above all of which are intended to fall within the scope of the invention as defined in the claims that follow.

1. An assembly for allergy testing, the assembly comprising:
at least one apparatus having a plurality of head elements, each head element including four pressure tips interspersed alternatingly between four piercing tips and together arranged in a circular pattern within which circular pattern a fifth piercing tip is centrally located;
a lower tray including a plurality of well sections for retaining a plurality of allergens, the well sections including a plurality of wells corresponding to the plurality of head elements;
a cover for removable attachment to the lower tray; and
a top tray for retaining the apparatus,
wherein the cover, upon attachment to the lower tray, retains the top tray and the at least one apparatus therebetween.

2. The assembly as claimed in claim 1, wherein the apparatus includes two rows of head elements and the total number of head elements is eight.

3. The assembly as claimed in claim 1, wherein the apparatus includes three rows of head elements and the total number of head elements is twelve.

4. The assembly as claimed in claim 3, wherein a middle one of the three rows is displaced relative to an outer two of the three rows.

5. The assembly as claimed in claim 1, wherein the apparatus includes two rows of head elements.

6. (canceled)

7. The assembly as claimed in claim 1, wherein the pressure tips include a rounded end.

8. A kit for allergy testing, the kit comprising:
at least one apparatus having a plurality of head elements, each head element capable of providing four pressure tips interspersed alternatingly between four piercing tips and together arranged in a circular pattern within which circular pattern a fifth piercing tip is centrally located;
a lower tray capable of providing a plurality of well sections for retaining a plurality of allergens, the well sections providing a plurality of wells corresponding to the plurality of head elements;
a cover capable of secure attachment to the lower tray; and
a top tray capable of securely situating the apparatus,
wherein the cover is capable of removable attachment to the lower tray and situating the top tray along with the apparatus therebetween.

9. A method of administering an allergy test by means of an apparatus having at least one head element including a plurality of piercing tips and a plurality of rounded pressure tips slightly shorter than the plurality of piercing tips, the method comprising:
placing the head element into at least one well containing an allergen solution to load the allergen solution onto the head element; and
administering the allergen solution to a skin surface of a patient by pressing the apparatus against the skin surface such that the rounded pressure tips of the head element contact with the skin immediately after the piercing tips of the head element punctures the skin of the patient and delivers the allergen solution,
wherein the pressure tips activate nerve tissue of the patient to suppress pain thereby resulting in a substantial reduction of pain during testing.
